(19) United States
(12) Patent Application Publication
(10) Pub. No.: US 2009/0130028 A1
(43) Pub. Date: May 21, 2009

(54) Cosmetic composition comprising a cationic polyurethane and a silicone, to be applied during hair dressing

(76) Inventors: Isabelle Rollat-Corvol, Paris (FR); Pascale Cothias, Montigny le Bretonneux (FR); Katarina Benabdillah, Le Plessie-Bouchard (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/230,877

(22) Filed: Sep. 5, 2008

Related U.S. Application Data
(63) Continuation of application No. 12/007,627, filed on Jan. 14, 2008, now abandoned.
(60) Provisional application No. 60/903,301, filed on Feb. 26, 2007.

(30) Foreign Application Priority Data
Jan. 12, 2007 (FR) 0752651

(51) Int. Cl. A61K 8/89; A61Q 5/06

(52) U.S. Cl. 424/47; 424/70.12

(57) ABSTRACT

The present disclosure relates to a cosmetic composition and hairstyling method comprising, in a cosmetically acceptable aqueous medium:
(i) at least one cationic polyurethane comprising at least one non-ionic unit derived from at least one polymer chosen from olefinic homopolymers and copolymers, and
(ii) at least one silicone chosen from polydialkyl siloxanes and organomodified polysiloxanes comprising at least one functional group chosen from poly(oxyalkylene), amine and alkoxy groups.

This application claims benefit of U.S. Provisional Application No. 60/903,301, filed Feb. 27, 2007, the contents of which are incorporated herein by reference. This application also claims benefit of priority under 35 U.S.C. § 119 to French Patent Application No. FR 0752651, filed Jan. 12, 2007, the contents of which are also incorporated herein by reference.

The present disclosure relates to new cosmetic compositions, such as hairstyling compositions, comprising the combination of at least one cationic polyurethane comprising non-ionic units derived from at least one polymer chosen from olefinic homopolymers and copolymers, and at least one specific silicone.

The use of elastic cationic polyurethanes in cosmetic compositions, such as hairstyling compositions, is known.

Thus, French patent application FR 2 815 350 describes cationic polyurethanes of the elastic type and their use for formulating hair sprays and hair styling compositions to enhance hair suppleness, i.e., making it possible to hold hair styles elastically, in a more natural way as compared to that obtained with usual fixing polymers.

French patent application FR 2 833 960 describes cosmetic styling compositions, for example hair styling compositions, such as styling shampoos, comprising a self-adhering cationic or amphoteric polyurethane. Such compositions may further comprise a silicone as an additive.

The present inventors have found that using an elastic cationic polyurethane comprising units derived from an olefinic homo- and/or copolymer in a hair styling composition may provide an excellent hold over time, but may lead to cosmetically poor properties and may be difficult to remove with shampoo.

Surprisingly and unexpectedly, the present inventors have discovered that combining certain silicones with these cationic polyurethanes comprising units derived from an olefinic homo- and/or copolymer makes it possible to formulate cosmetic hairstyling compositions resulting in good cosmetic properties, while being easily removable with shampoo, without affecting hair fixing and its ability to keep hair in place over time.

The present disclosure thus relates to a cosmetic composition, for example a hair styling composition, comprising, in a cosmetically acceptable medium, at least one cationic polyurethane comprising at least one unit derived from at least one polymer chosen from olefinic homopolymers and copolymers and at least one specific silicone.

The present disclosure also relates to such a composition further comprising a gas propellant and being in the form of an aerosol.

The present disclosure still further relates to a hairstyling method comprising applying onto the hair such a composition, then styling and drying the hair.

According to the present disclosure, the cosmetic composition comprises, in a cosmetically acceptable aqueous medium:

(i) at least one cationic polyurethane comprising at least one non-ionic unit derived from at least one polymer chosen from olefinic homopolymers and copolymers, and

(ii) at least one silicone chosen from polydialkyl siloxanes and organomodified polysiloxanes comprising at least one functional group chosen from poly(oxyalkylene), amino and alkoxy groups.

### Cationic Polyurethanes

The elastic cationic polyurethane comprising at least one non-ionic unit, derived from at least one polymer chosen from olefinic homopolymers and copolymers is the first component of the compositions disclosed herein.

In at least one embodiment, the at least one cationic polyurethane to be suitably used in the present disclosure comprises:

(a) at least one cationic unit derived from at least one compound, such as a tertiary or a quaternary amine, comprising at least two labile hydrogen-containing reactive functions,

(b) at least one non-ionic unit derived from non-ionic polymers carrying labile hydrogen-containing reactive functions at their ends, wherein at least one of the (b) units, for example at least 50% by weight of the total weight of the (b) units or for example all the (b) units, is at least one (b1) unit derived from at least one olefinic homo- or copolymer carrying labile hydrogen-containing reactive functions at its ends, and

(c) at least one unit derived from at least one diisocyanate.

As used herein, “cationic unit” is intended to mean any unit that, either due to its own chemical nature, or because of its environment and/or the pH value by which it is surrounded is in a cationic form.

As used herein, “labile hydrogen-containing reactive functions” means functions that are able, after the departure of a hydrogen atom, to form covalent bonds with the isocyanate functions of the compounds forming the (c) units. Suitable examples of such functions include, but are not limited to, hydroxyl, primary amine (—NH₂) or secondary amine (—NHR), or thiol (—SH) groups.

In at least one embodiment, polycondensation of compounds carrying these labile hydrogen-containing reactive functions with diisocyanates results in polyurethanes, polyureas or polythiourethanes, depending on the nature of the labile hydrogen-carrying reactive functions (—OH, —NH₂, —NHR or —SH), respectively. For greater convenience, all these polymers are intended to be encompassed in the present disclosure within the polyurethane class. In at least one embodiment, the polymers of the present disclosure are authentic polyurethanes.

In an embodiment when the at least one tertiary or quaternary amine forming the (a) units carries more than two labile hydrogen-containing functions, the resulting polyurethanes may have a branched structure.

In another embodiment of the polyurethane of the present disclosure, the at least one tertiary or quaternary amine forming the at least one cationic (a) unit only comprises two labile hydrogen-containing reactive functions and consequently the polyurethanes resulting from the polycondensation may have a substantially linear structure.

In yet another embodiment, it is also possible to use a mixture comprising difunctional amines comprising a small amount of amines carrying more than two labile hydrogen-containing reactive functions.

In at least one embodiment, the at least one tertiary or quaternary amine forming the cationic (a) unit is chosen from compounds corresponding to one or more of the following formulas:

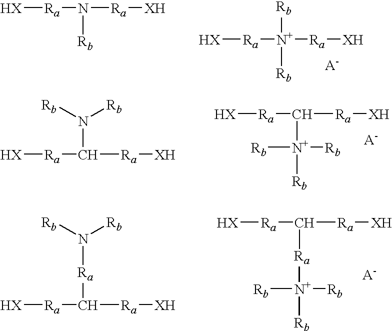

wherein

each Rₐ is independently chosen from linear or branched C₁₋₆ alkylene, C₃₋₆ cycloalkylene, and arylene groups, where all of them may be substituted with at least one halogen atom and comprise at least one heteroatom chosen from O, N, P and S;

each R_{b} is independently chosen from C₁₋₆ alkyl groups, C₃₋₆ cycloalkyl groups, and aryl groups, where all of them may be substituted with at least one halogen atom and comprise at least one heteroatom chosen from O, N, P and S;

each X is independently chosen from oxygen and sulfur atoms, and from NH and NR_{c} groups, wherein R_{c} is a C₁₋₆ alkyl group; and

A⁻ is a physiologically acceptable counter-ion.

In at least one embodiment, N-methyldiethanol amine and N-tert-butyldiethanol amine are tertiary amines that are used for producing said cationic polyurethanes.

Tertiary and quaternary amines forming the cationic (a) units of the polyurethanes of the present disclosure may also be tertiary and/or quaternary amine function-containing polymers, carrying labile hydrogen-containing reactive functions at their ends. The weight average molecular weight of such tertiary and/or quaternary amine function-containing polymers may, in at least one embodiment, range from 400 to 10,000.

As suitable non-limiting examples of such amine function-containing polymers, polyesters resulting from the polycondensation of N-methyldiethanol amine and adipic acid may be mentioned.

When the amines forming the cationic (a) units are tertiary amine function compounds, all or part of these amine functions are neutralized with a suitable neutralizing agent chosen from physiologically acceptable organic or mineral acids. Hydrochloric acid or acetic acid may be mentioned as non-limiting acid examples.

The second type of unit forming the polyurethanes of the present disclosure includes, in at least one embodiment, at least one macromolecular unit, herein called (b) unit(s), derived from non-ionic polymers carrying labile hydrogen-containing reactive functions at their ends, such as those with a glass transition temperature (Tg) lower than 10° C., as measured by differential enthalpy analysis.

In one embodiment according to the present disclosure, at least one (b1) unit is derived from at least one polymer chosen from olefinic homopolymers and copolymers.

In at least one embodiment, the polyurethane viscoelastic properties may be advantageous when (b) units are derived from polymers having a glass transition temperature lower than 0° C., for example lower than −10° C.

These polymers may have a weight average molecular weight ranging from 400 to 10,000, for example from 1000 to 5000.

Non-ionic polymers that can form the at least one (b2) non-ionic unit(s) different from the at least one (b1) non-ionic unit(s) derived from at least one polymer chosen from olefinic homopolymers and copolymers, may be chosen from polyethers, polyesters, polysiloxanes, polycarbonates and fluorinated polymers.

In at least one embodiment, polymers that can form said at least one (b) non-ionic unit(s) are only chosen from olefinic homo- and copolymers.

Examples of olefinic polymers having labile hydrogen-containing reactive groups on their terminal ends, to be suitably used in the present disclosure, include, but are not limited to, ethylene, propylene, 1-butylene, 2-butylene, isobutylene, 1,2-butadiene, 1,4-butadiene and isoprene random or block homopolymers and copolymers.

Butadiene and isoprene homo- and copolymers may be partially or fully hydrogenated.

In at least one embodiment, the polymers are copolymers of ethylene and butylene, polybutadienes and hydrogenated polybutadienes carrying on their terminal ends labile hydrogen-containing reactive groups, for example hydroxyl groups. In a further embodiment, these polymers are 1,2- and/or 1,4-polybutadienes.

Such polymers are commercially available for example under the trade name KRATON® L, more particularly KRATON® L 2203 (hydrogenated polybutadiene diol) from the KRATON polymers company, KRASOL LBH® and LBHP®, especially KRASOL LBHP® 2000 (polybutadiene diol) from the SARTOMER company and GI® 3000 (copolymer of ethylene and butylene) from the NISSO CHEMICAL company.

The at least one diisocyanates forming the (c) units include aliphatic, alicyclic or aromatic diisocyanates.

In at least one embodiment, the diisocyanates are chosen from methylenediphenyl diisocyanate, methylenecyclohexane diisocyanate, isophorone diisocyanate, toluene diisocyanate, naphthalene diisocyanate, butane diisocyanate and hexyl diisocyanate. These diisocyanates may be used alone or as a mixture of two or more diisocyanates. In a further embodiment, said diisocyanate is isophorone diisocyanate.

As previously mentioned, cationic polyurethanes of the present invention may contain, in addition to (a), (b1) and (c) units, a certain content of (b2) units derived from monomeric, non-ionic compounds comprising at least two labile hydrogen functions, different from the compounds leading to the (b1) units.

These (b2) units may, for example, be derived from C₁-C₁₂ diols, for example from neopentyl glycol, hexaethylene glycol, 1,2-ethanediol, 1,2-propanediol and 1,3-propanediol or from C₁-C₆ aminoalcohols, for example from aminoethanol.

In at least one embodiment, the cationic polyurethanes of the present disclosure are elastic.

In another embodiment of the disclosure, said at least one cationic polyurethane does not comprise any further unit in addition to the (a), (b) and (c) units. The polyurethane (A) described in the examples is a polyurethane corresponding to such definition.

In an alternative embodiment, the cationic polyurethane comprises further units, in addition to the (a), (b) and (c) units. The polyurethane (B) described in the examples is a polyurethane corresponding to such definition.

A physical parameter characterizing the viscoelastic properties of the above cationic polyurethanes is their tensile recovery. Such recovery is determined by a tensile creep test consisting of rapidly stretching a specimen to a predetermined degree of elongation, then releasing the stress, and lastly measuring the specimen length.

The creep test used to characterize the cationic polyurethanes with elastic character of the present disclosure is performed as follows:

The specimen used is a film of polyurethane 500±50 mm-thick, cut into 80 mm×15 mm strips. This copolymer film is obtained by drying at a temperature of 22±2° C. under a 50±5% relative humidity, a 3% by weight solution or dispersion of said polyurethane in water and/or in ethanol.

Each strip is fixed between two jaws, spaced apart from each other by 50±1 mm, and is stretched at a speed of 20 mm/minute (under the above mentioned temperature and relative humidity conditions) up to a 50% elongation (εₘₐₓ), that is to say until a strip is obtained, which size corresponds to 1.5 times its initial length. The stress is then released by setting a return speed equal to the tensile speed, i.e., 20 mm/minute, and the specimen elongation is then measured (as expressed in % relative to the initial length) immediately once it has returned to a zero load (εᵢ).

The instantaneous recovery (Rᵢ) is calculated using the following equation:

Rᵢ (%)=((εₘₐₓ−εᵢ)/εₘₐₓ)×100

In at least one embodiment, the elastic cationic polyurethanes of the present disclosure have an instantaneous recovery (Rᵢ), such as measured in the above stated conditions and ranging from 5% to 95%, for example from 20% to 90% or from 35 to 85%.

The glass transition temperature (Tg) of the non-ionic polymers forming the (b) units and of the cationic polyurethanes of the present disclosure is measured by means of a differential enthalpy analysis (DSC, differential scanning calorimetry) according to ASTM D3418-97 standard.

In at least one embodiment, the elastic cationic polyurethanes of the present disclosure present at least two glass transition temperatures, at least one of which is lower than 10° C., such as lower than 0° C. or lower than −10° C., the other one being at least higher than or equal to the room temperature (20° C.).

The instantaneous recovery and therefore the viscoelastic properties of the polyurethanes of the present disclosure depend on the contents of the various (a), (b1), (b2) and (c) monomer units.

In at least one embodiment, the (a) unit is present in an amount sufficient to provide the polymers with the positive charge responsible for their good affinity for keratinic substrates and the (b) unit is present in an amount sufficient for the polyurethanes to have at least one glass transition temperature lower than 10° C. and not to form brittle films.

In at least one embodiment, the (a) unit(s) is/are present in an amount ranging from 0.1 to 90% by weight relative to the total weight of the polyurethane units, for example from 1 to 30%, and for example from 5 to 25% and for example from 5 to 10% by weight; the at least one (b1) unit(s) from 10 to 99.9%, for example from 20 to 99% or from 30 to 85% by weight; and the (b2) unit(s) from 0 to 50% by weight, for example from 0 to 30% by weight relative to the total weight of the polyurethane units. In a further embodiment, the polyurethanes of the present disclosure do not comprise any (b2) unit.

In at least one embodiment, the (c) unit(s) is/are present in an amount ranging from 1 to 60% relative to the polyurethane unit total weight, for example from 5 to 50% or from 10 to 40%.

In at least one embodiment, the (c) unit(s) is/are present in a substantially stoichiometric amount as compared to the sum of (a) and (b) units. Obtaining polyurethanes with high molecular weights may require a number of isocyanate functions almost identical to the number of labile hydrogen functions. The person skilled in the art will be able to choose an optional molar excess of the one function or the other, to adjust the molecular weight to the expected value.

The amount of polyurethane present in a cosmetic composition of the present disclosure of course depends on the composition type and the required properties, and may vary within a very broad range, such as an amount ranging from 0.01 to 40% by weight relative to the weight of the final cosmetic composition, for example from 0.05 to 20% or from 0.1 to 10% by weight.

### Silicones

The second main component of the compositions of the present disclosure is a silicone chosen from polydialkyl siloxanes, such as polydimethyl siloxanes (PDMS), and organomodified polysiloxanes comprising at least one functional group chosen from poly(oxyalkylene), amino- and alkoxy groups.

Silicones to be used as additives in the cosmetic compositions of the disclosure are volatile or non volatile, cyclic, linear or branched silicones, modified with organic groups, or not, and having a viscosity ranging from 5×10⁻⁶ to 2.5 m²/s at 25° C., for example from 1×10⁻⁵ to 1 m²/s.

Silicones to be used according to the present disclosure may be soluble or insoluble in the composition and, in one embodiment, may be polyorganosiloxanes insoluble in the composition of the disclosure. They may be present, for example, in the form of oils, waxes, resins or gums.

Organopolysiloxanes are defined in more detail by Walter NOLL in “Chemistry and Technology of Silicones” (1968) Academie Press. They may be volatile or not.

When they are volatile, silicones are chosen, in at least one embodiment, from those having a boiling point ranging from 60° C. to 260° C., for example from the following:

(i) cyclic polydialkylsiloxanes comprising from 3 to 7, for example 4 or 5, silicon atoms. Suitable examples thereof include, but are not limited to, octamethyl cyclotetrasiloxane marketed, for example, under the trade name “VOLATILE SILICONE® 7207” by UNION CARBIDE or “SILBIONE® 70045 V 2” by RHODIA, decamethyl cyclopentasiloxane marketed under the trade name “VOLATILE SILICONE® 7158” by UNION CARBIDE, “SILBIONE® 70045 V 5” by RHODIA, as well as mixtures thereof.

Cyclocopolymers of the dimethyl siloxane/methylalkyl siloxane type may also be mentioned, for example, such as “SILICONE VOLATILE® FZ 3109” marketed by the UNION CARBIDE company, having the following formula:

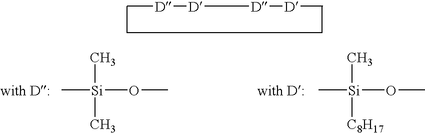

Mixtures of cyclic polydialkyl siloxanes with organic compounds derived from silicon may also be mentioned, such as the octamethyl cyclotetrasiloxane and tetratrimethylsilyl pentaerythritol mixture (50:50) and the octamethyl cyclotetrasiloxane and oxy-1,1′-(hexa-2,2,2′,2′,3,3′-trimethylsilyloxy) bis-neopentane mixture;
(ii) linear volatile polydialkyl siloxanes having from 2 to 9 silicon atoms and the viscosity of which is lower than or equal to 5×10⁻⁶ m²/s at 25° C., for example decamethyl tetrasiloxane marketed, for example, under the trade name “SH 200” by the TORAY SILICONE company. Silicones belonging to this class are also, for example, described in the article published in Cosmetics and Toiletries, Vol. 91, Jan. 76, P. 27-32—TODD & BYERS “Volatile Silicone fluids for cosmetics.”

In at least one embodiment, non volatile polydialkyl siloxanes are used, for example polydiaryl siloxanes and polyalkylaryl siloxanes, gums and polydialkyl siloxane resins, polyorganosiloxanes modified with organofunctional groups as well as mixtures thereof.

In another embodiment, said polyalkyl siloxanes are (C₁-C₈)polydialkyl siloxanes, such as (C₁-C₄)polydialkyl siloxanes.

In at least one embodiment, these silicones are chosen from polydialkyl siloxanes, from which polydimethyl siloxanes with trimethylsilyl end groups may be mentioned by way of non-limiting example. Silicone viscosity is measured at 25° C. according to ASTM 445 standard, Appendix C.

These polydialkyl siloxanes encompass, as non-limiting examples, the following commercial products:
SILBIONE® oils of 47 and 70 047 series or MIRASIL® oils marketed by RHODIA, such as for example fluid 70 047 V 500 000;
oils of MIRASIL® series marketed by the RHODIA company;
oils of the 200 series from the DOW CORNING company, such as DC200 (viscosity 60,000 mm²/s);
VISCASIL® oils from GENERAL ELECTRIC and certain oils of the SF (SF 96, SF 18) series from GENERAL ELECTRIC.

Dimethylsilanol end group-containing polydimethyl siloxanes, known under the name dimethiconol (CTFA) may also be mentioned, such as oils of the 48 series from the RHODIA company.

This polydialkyl siloxane class also includes products marketed under the trade names “ABIL WAX® 9800 and 9801” by the GOLDSCHMIDT company, which are (C₁-C₂₀) polydialkyl siloxanes.

Examples of silicone gums to be suitably used according to the present disclosure include, but are not limited to, polydialkyl siloxanes, such as polydimethyl siloxanes having high number average molecular weights ranging from 200,000 and 1,000,000 used either alone or in combination in a solvent. In at least one embodiment, this solvent may be chosen from volatile silicones, polydimethyl siloxanes (PDMS) oils, polyphenylmethyl siloxanes (PPMS) oils, isoparaffins, polyisobutylenes, methylene chloride, pentane, dodecane, tridecane, and mixtures thereof.

Products that may be used according to the present disclosure are mixtures such as, but not limited to:

mixtures formed from an end chain-hydroxylated polydimethyl siloxane also called dimethiconol (CTFA) and a cyclic polydimethyl siloxane, also called cyclomethicone (CTFA), such as the Q2 1401 product marketed by the DOW CORNING company;

mixtures formed from a polydimethyl siloxane gum and a cyclic silicone, such as the SF 1214 Silicone Fluid from the GENERAL ELECTRIC company, this product being a SF 30 gum corresponding to a dimethicone, having a number average molecular weight of 500,000, solubilized in the SF 1202 Silicone Fluid corresponding to decamethyl cyclopentasiloxane;

mixtures formed from two PDMS with different viscosities, for example from a PDMS gum and a PDMS oil, such as the SF 1236 product from GENERAL ELECTRIC. SF 1236 is a mixture of a SE 30 gum as defined above with a viscosity of 20 m²/s and a SF 96 oil with a viscosity of 5.10⁻⁶ m²/s. Such product comprises, for example, 15% of SE 30 gum and 85% of SF 96 oil.

In at least one embodiment, organopolysiloxane resins to be used according to the present disclosure are crosslinked siloxane systems comprising units:

R₂SiO_{2/2}, R₃SiO_{1/2}, RSiO_{3/2} and SiO_{4/2}, wherein R represents an alkyl group comprising from 1 to 16 carbon atoms. Amongst these products, non-limiting mention may be made of those wherein R represents a lower C₁-C₄ alkyl group, such as a methyl group.

These resins also include the product marketed under the trade name “DOW CORNING 593” or those marketed under the trade names “SILICONE FLUID SS 4230 and SS 4267” by the GENERAL ELECTRIC company and which are dimethyl/trimethyl siloxane-structured silicones.

Resins of the trimethyl siloxysilicate type marketed, for example, under the trade names X22-4914, X21-5034 and X21-5037 by the SHIN-ETSU company may also be mentioned.

In one embodiment, polydiaryl siloxanes may be polydiphenyl siloxanes. In another embodiment, polyalkylaryl siloxanes may be chosen from linear and/or branched polydimethyl/methylphenyl siloxanes and polydimethyl/diphenyl siloxanes having viscosities ranging from 1×10⁻⁵ to 5×10⁻² m²/s at 25° C.

Suitable examples of such polyalkylaryl siloxanes include, but are not limited to, products marketed under the following trade names:
SILBIONE® oils of the 70 641 series from RHODIA;
oils of RHODORSIL® 70 633 and 763 series from RHODIA;
DOW CORNING 556 COSMETIC GRAD FLUID from DOW CORNING;
silicones of the PK series from BAYER, such as the PK20 product;
silicones of the PN, PH series from BAYER, such as PN1000 and PH1000 products; and
certain oils of the SF series from GENERAL ELECTRIC, such as SF 1023, SF 1154, SF 1250, and SF 1265.

In at least one embodiment, organomodified silicones to be suitably used according to the disclosure are silicones such as previously defined, and comprising in their structure at least one organofunctional group bound through a hydrocarbon group.

Examples of organomodified silicones to be suitably used according to the disclosure include, but are not limited to, polyorganosiloxanes comprising:
polyethyleneoxy and/or polypropyleneoxy groups optionally comprising C₆-C₂₄ alkyl groups, such as products called dimethicone copolyol marketed by the DOW CORNING company under the trade name DC 1248 or SILWET® L 722, L 7500, L 77, L 711 oils from the UNION CARBIDE company and (C₁₂)alkyl methicone copolyol marketed by the DOW CORNING company under the trade name Q2 5200;

amine groups, substituted or not, such as the products marketed under the trade name GP 4 Silicone Fluid and GP 7100 by the GENESEE company, or the products marketed under the trade names Q2 8220 and DOW CORNING 929 or 939 by the DOW CORNING company. For example, substituted amine groups are from C₁-C₄ aminoalkyl groups. Such amino silicones may carry alkoxy groups, for example methoxy groups, such as BELSIL ADM LOG 1 silicone marketed by the WACKER company;

alkoxyl groups, such as the product marketed under the trade name “SILICONE COPOLYMER F-755” by SWS SILICONES and ABIL WAX® 2428, 2434 and 2440 by the GOLDSCHMIDT company.

The silicones such as described above may be used either alone or in combination, and are present in an amount ranging from 0.01 to 20% by weight relative to the total weight of the composition, for example from 0.1 to 5% by weight.

### Cosmetic Additives and Solvents

The cosmetically acceptable aqueous medium may comprise various additives and solvents commonly used in the cosmetic field such as surfactants, gelling agents and/or thickeners, organic solvents, fragrances, mineral, vegetable and/or synthetic oils or waxes, fatty acid esters, pigments and dyes, mineral or organic particles, pH stabilizing agents, preserving agents and UV absorbers.

In at least one embodiment, surfactants to be used in the composition of the present disclosure may be anionic, non-ionic, amphoteric or cationic surfactants, or mixtures thereof.

Examples of suitable anionic surfactants to be used either alone or in combination in the context of the present disclosure include, but are not limited to, salts, such as alkaline metal salts such as sodium salts, ammonium salts, amine salts, aminoalcohol salts or alkaline-earth metal salts, for example, magnesium salts, of the following compounds: alkyl sulfates, alkyl ethersulfates, alkyl amidoethersulfates, alkyl-aryl polyethersulfates, monoglyceride sulfates; alkyl sulfonates, alkyl amidesulfonates, alkyl-aryl sulfonates, α-olefin sulfonates, paraffin sulfonates; alkyl sulfosuccinates, alkyl ethersulfosuccinates, alkylamide sulfosuccinates; alkyl sulfoacetates; acyl sarconisates; and acylglutamates, the alkyl and acyl groups of all these compounds comprising from 6 to 24 carbon atoms and the aryl group corresponding, for example, to a phenyl or benzyl group.

In at least one embodiment, polyglycoside carboxylic acid and C₆-C₂₄ alkyl esters may also be used in the context of the present disclosure, such as alkyl glucoside citrates, alkyl polyglycoside tartrates and alkyl polyglycoside sulfosuccinates; as well as alkyl sulfosuccinamates, acyl isethionates and N-acyl taurates, the alkyl or acyl group of all these compounds comprising from 12 to 20 carbon atoms. As further non-limiting examples of anionic surfactants to be suitably used, acyl lactylates the acyl group of which comprises from 8 to 20 carbon atoms may also be mentioned.

Moreover, alkyl-D-galactoside uronic acids and salts thereof may also be mentioned, as well as polyoxyalkylene (C₆-C₂₄)alkylether carboxylic acids, polyoxyalkylene (C₆-C₂₄)alkyl(C₆-C₂₄)arylether carboxylic acids, polyoxyalkylene (C₆-C₂₄)alkylamidoether carboxylic acids and salts thereof, such as those comprising from 2 to 50 ethylene oxide groups, and mixtures thereof.

Amongst the above mentioned anionic surfactants, in at least one embodiment according to the present disclosure, the anionic surfactants to be used herein may be chosen from (C₆-C₂₄)alkyl sulfates, (C₆-C₂₄)alkyl ethersulfates, (C₆-C₂₄)alkyl ethercarboxylates and mixtures thereof, for example ammonium lauryl sulfate, sodium lauryl sulfate, magnesium lauryl sulfate, sodium lauryl ethersulfate, ammonium lauryl ethersulfate and magnesium lauryl ethersulfate.

Non-ionic surfactants to be used in the context of the present disclosure are also compounds that are well known to those skilled in the art (for a review thereof, see, for example, “Handbook of Surfactants” M. R. PORTER, Blackie & Son Editor (Glasgow and London), 1991, pp 116-178). In at least one embodiment, they may be chosen from alcohols, alpha-diols, (C₁-C₂₀)alkyl phenols or polyethoxylated, polypropoxylated or polyglycerolated fatty acids, having a fatty chain comprising for example from 8 to 18 carbon atoms, wherein the number of ethylene oxide or propylene oxide groups may range from 2 to 50 and the number of glycerol groups may range from 2 to 30. Also to be mentioned as non-limiting examples are copolymers of ethylene oxide and propylene oxide, condensation products of ethylene oxide and propylene oxide on fatty alcohols; polyethoxylated fatty amides comprising, for example, from 2 to 30 moles of ethylene oxide; polyglycerolated fatty amides comprising on average from 1 to 5 glycerol groups, such as from 1.5 to 4; polyethoxylated fatty amines comprising, for example, from 2 to 30 moles of ethylene oxide; sorbitane fatty acid esters ethoxylated with from 2 to 30 moles of ethylene oxide; sucrose fatty acid esters, polyethylene glycol fatty acid esters, (C₆-C₂₄)alkyl polyglucosides, (C₆-C₂₄)N-alkyl glucamine derivatives, amine oxides such as (C₁₀-C₁₄)alkyl amine oxides or (C₁₀-C₁₄)N-acyl aminopropylmorpholine oxides; and mixtures thereof.

In at least one embodiment, amongst the previously mentioned non-ionic surfactants, the (C₆-C₂₄)alkyl polyglycosides are used, for example decyl polyglucoside.

Examples of amphoteric surfactants to be suitably used in the present disclosure include, but are not limited to, secondary or tertiary aliphatic amine derivatives, wherein the aliphatic group is a linear or a branched chain comprising from 8 to 22 carbon atoms and containing, at least one hydrosolubilizing anionic group such as, for example, a carboxylate, sulfonate, sulfate, phosphate or phosphonate group; (C₈-C₂₀)alkyl betaines, sulfobetaines, (C₈-C₂₀)alkyl (C₆-C₈)amidoalkyl betaines or (C₈-C₂₀)alkyl (C₆-C₈)amidoalkyl sulfobetaines, as well as mixtures thereof, may also be mentioned.

Amongst the amine derivatives, products marketed under the trade name MIRANOL® may be mentioned as non-limiting examples, such as those described in U.S. Pat. Nos. 2,528,378 and 2,781,354 and classified in the CTFA dictionary, third Edition, 1982, under the names amphocarboxyglycinate and amphocarboxypropionate having, respectively, the following structures (1) and (2):

R₂—CONHCH₂CH₂—N⁺(R₃)(R₄)(CH₂COO⁻) (1)

wherein:

R₂ is chose from an alkyl group derived from a R₂—COOH acid present in hydrolyzed coconut oil, and from heptyl, nonyl and undecyl groups,

R₃ is a beta-hydroxyethyl group, and

R₄ is a carboxymethyl group; and

R₂—CONHCH₂CH₂—N(B)(C) (2)

wherein:

B is —CH₂CH₂OX′,

C is —(CH₂)_{z}—Y′, wherein z is equal to 1 or 2,

X′ is chosen from —CH₂CH₂—COOH groups and hydrogen atoms,

Y′ is chosen from —COOH and —CH₂—CHOH—SO₃H groups,

R₂ is chosen from alkyl groups of a R₂—COOH acid present in hydrolyzed coconut oil or linseed oil, alkyl groups, such as C₁₋₇ alkyl groups and their iso-forms, and unsaturated C₁₇ groups.

These compounds are, for example, classified in the CTFA dictionary, 5th Edition, 1993, under the names disodium cocoamphodiacetate, disodium lauroamphodiacetate, disodium capryl amphodiacetate capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

The cocoamphodiacetate marketed under the trade name MIRANOL® C2M concentrated by the RHODIA company is a suitable non-limiting example thereof.

In one embodiment, as suitable amphoteric surfactants, (C₈-C₂₀)alkyl betaines are used, such as coco betaine, (C₈-C₂₀)alkyl (C₆-C₈)amidoalkyl betaines such as cocamido betaine, alkyl amphodiacetates such as disodium cocoamphodiacetate, and mixtures thereof.

Moreover, the composition of the present disclosure may further comprise at least one cationic surfactant, such as those that are well known to those skilled in the art, such as salts of primary, secondary or tertiary fatty amines, optionally polyoxyalkylenated, quaternary ammonium salts such as tetraalkylammonium, alkylamidoalkyl trialkylammonium, trialkylbenzylammonium, trialkylhydroxyalkylammonium or alkylpyridinium chlorides or bromides, imidazoline derivatives; or amine oxides of cationic nature.

The previously described non-ionic, amphoteric, anionic and cationic surfactants may be used either alone or in combination. In at least one embodiment, the at least one surfactant is present in an amount ranging from 0.01 to 60% by weight relative to the composition total weight, for example from 0.1 to 30% or from 1 to 20% by weight Gelling agents and/or thickeners that may be suitably used in the compositions of the present disclosure are well known in the art and may, for example, but not by way of limitation, be chosen from carboxyvinyl polymers and copolymers, (alkyl)acrylic polymers and copolymers, (alkyl)acrylamide polymers and copolymers, poly(oxyalkylene) glycols, poly(oxyalkylene) glycol esters, alginates, biosaccharides, polysaccharides such as cellulose and starch derivatives, naturally occurring gums such as xanthan gum, guar gum, locust bean gum, scleroglucans, chitin and chitosan derivatives, carrageenans, clays, and mixtures thereof.

Examples of gelling agents, such as those in an aqueous phase, include, but are not limited to, SEPIGEL® 305 marketed by the SEPPIC company, FUCOGEL® 1000 PP marketed by the SOLABIA company, SYNTHALEN® K marketed by the 3VSA company, LUVISKOL® VA 64 P marketed by the BASF company, HOSTACERIN® AMPS marketed by the CLARIANT company, PEMULEN® TR1 marketed by the GOODRICH company, LUBRAGEL® MS marketed by the GUARDIAN company, SATIAGEL® KSO marketed by DEGUSSA and KELTROL® marketed by the KELCO company.

In at least one embodiment, the gelling agent is present in an amount ranging from 0.1 to 15% by weight of the composition, for example from 0.5 to 10%.

The compositions of the present disclosure may also comprise fatty components such as mineral, vegetable, animal and synthetic oils, waxes, fatty esters, fatty alcohols, and fatty acids.

Suitable examples of oils to be used in the composition of the disclosure include, but are not limited to:

animal-based hydrocarbon oils, such as perhydrosqualene;

vegetable-based hydrocarbon oils, such as liquid triglycerides of fatty acids comprising from 4 to 10 carbon atoms such as triglycerides of the heptanoic or octanoic acids, or for example sunflower oil, corn oil, soja bean oil, pumpkin oil, grape seed oil, sesame oil, nut oil, apricot kernel oil, macadamia nut oil, arara oil, castor oil, avocado oil, triglycerides of caprylic/capric acids such as those marketed by the Stearineries Dubois company or those sold under the names Miglyol® 810, 812 and 818 by the Dynamit Nobel company, jojoba oil, shea butter oil;

linear or branched, mineral or synthetic hydrocarbons, such as volatile or non volatile paraffin oils, and their derivatives, petrolatum, polydecenes, hydrogenated polyisobutene such as Parleam®; isoparaffines such as isohexadecane and isodecane;

partly hydrocarbon-based and/or silicone-based fluorinated oils, such as those described in Japanese patent application JP-A-2-295912; fluorinated oils also encompass perfluoromethyl cyclopentane and perfluoro-1,3 dimethylcyclohexane, sold under the names “FLUTEC® PC1” and “FLUTEC® PC3” by the BNFL Fluorochemicals company; perfluoro-1,2-dimethyl cyclobutane; perfluoroalkanes such as dodecafluoropentane and tetradecafluorohexane, sold under the names “PF 5050®” and “PF 5060®” by the 3M company, or bromoperfluorooctyle sold under the trade name “FORALKYL®” by the Atochem company; nonafluoromethoxybutane and nonafluoroethoxyisobutane; and perfluoromorpholine derivatives, such as 4-trifluoromethyl perfluoromorpholine sold under the trade name “PF 5052®” by the 3M company;

In at least one embodiment, the at least one wax is chosen from Carnauba waxes, Candellila waxes, and Alfa waxes, paraffins, ozokerites, vegetable waxes such as olive tree wax, rice wax, hydrogenated jojoba wax or flower absolute waxes such as Ribes nigrum (blackcurrant) flower wax sold by the BERTIN company (France), and animal waxes such as beeswax, or modified beeswaxes (cerabellina); other non-limiting examples of waxes or wax-based raw materials to be used according to the present disclosure are also marine waxes such as the one sold by the SOPHIM company under the reference M82, polyethylene waxes or polyolefins in general.

In at least one embodiment, saturated or unsaturated fatty acids are chosen from myristic acid, palmitic acid, stearic acid, behenic acid, oleic acid, linoleic acid, linolenic acid and isostearic acid.

In at least one embodiment, the fatty esters are chosen from carboxylic acid esters, for example mono, di, tri or tetracarboxylic esters.

In at least one embodiment, the carboxylic acid esters are saturated or unsaturated, linear or branched, C₁-C₂₆ aliphatic acid esters and saturated or unsaturated, linear or branched, C₁-C₂₆ aliphatic alcohol esters, wherein the total number of the ester carbon atoms is equal to or higher than 10.

Suitable examples of monoesters to be mentioned include, but are not limited to, dihydroabietyl behenate; octyidodecyl behenate; isocetyl behenate; cetyl lactate; C₁₂-C₁₅ alkyl lactate, isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyidodecyl erucate; oleyl erucate; ethyl and isopropyl palmitates, ethyl-2-hexyl palmitate, 2-octyldecyl palmitate, alkyl myristates, such as isopropyl, butyl, cetyl, 2-octyldodecyl myristate, hexyl stearate, butyl stearate, isobutyl stearate; and dioctyl malate, hexyl laurate, 2-hexyldecyl laurate.

C₄-C₂₂ di- or tricarboxylic acid and C₁-C₂₂ alcohol esters may also be used, as well as mono-, di- or tricarboxylic acid esters and di-, tri-, tetra- or pentahydroxy C₂-C₂₆ alcohol esters.

Further non-limiting examples to be mentioned are diethyl sebacate; diisopropyl sebacate; diisopropyl adipate; di n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetraerygonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisanonate; and polyethylene glycol distearates.

In at least one embodiment, the at least one ester is chosen from ethyl and isopropyl palmitates, ethyl-2-hexyl palmitate, 2-octyldecyl palmitate, alkyl myristates, such as isopropyl, butyl, cetyl, 2-octyldodecyl myristate, hexyl stearate, butyl stearate, isobutyl stearate; dioctyl malate, hexyl laurate, 2-hexyldecyl laurate and isononyl isononanate, and cetyl octanoate.

Suitable fatty alcohols include for example, but are not limited to, saturated or unsaturated, linear or branched fatty alcohols comprising from 8 to 26 carbon atoms, such as cetyl alcohol, stearyl alcohol and mixtures thereof (cetylstearyl alcohol), octyldodecanol, 2-butyloctanol, 2-hexyldecanol, 2-undecylpentadecanol, oleic alcohol or linoleic alcohol.

The fatty components may be present in an amount ranging from 0.01 to 50% by weight of the total composition, for example from 1 to 30% or from 2 to 20%.

The cosmetically acceptable aqueous medium of the composition, in addition to water, may further comprise at least one organic solvent.

The at least one organic solvent may be chosen from C₁-C₆ alcohols, for example alkanols, such as ethanol, propanol and isopropanol, alkanediols such as propylene glycol and pentanediols, benzyl alcohol, C₅-C₁₀ alkanes, acetone, methyl ethylcetone, methyl acetate, butyl acetate, ethyl acetate, dimethoxyethane, diethoxyethane and mixtures thereof.

The organic solvent may be present in an amount ranging from 0.5 to 80% by weight of the composition total weight, for example from 1 to 50% by weight.

The person skilled in the art will be able to add some additives without affecting the properties of the compositions of the disclosure.

The compositions of the present disclosure may be in the form of hairstyling compositions which may be rinsed off, such as styling shampoos, or not rinsed off, such as styling lotions, foams or gels.

In at least one embodiment, they may be in the form of a styling lotion or a styling gel.

In another embodiment, they also may be in the form of an aerosol. In that case, the composition will also comprise a propellant. As is well known, said propellant may, for example, be a gas or a mixture of compressed or liquefied gases, that may optionally be dissolved in the composition. Examples of suitable gas propellants encompass, but are not limited to, air, carbon dioxide, nitrogen, dimethyl ether, hydrocarbons such as propane, n-butane, isobutane or isopentane and halogenated hydrocarbons, such as fluorinated hydrocarbons. In another embodiment, silicones that are present in the compositions of the disclosure, may be initially introduced into or blend with the composition immediately prior to being applied.

In one embodiment wherein organomodified silicones by alkoxy groups are used, the silicone will be blended before the application.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below.

### FORMULATION EXAMPLES

### Example 1

### Styling Lotion

| | |
|---|---|
| Cationic polyurethane containing a polyolefin sequence¹ | 2% a.m. |
| DC 939 EMULSION² | 1% a.m. |
| DOW CORNING | |
| Demineralized water | Qs 100% |

### Example 2

### Styling Gel

| | |
|---|---|
| Cationic polyurethane containing a polyolefin sequence¹ | 6% a.m. |
| JAGUAR HP 105 (RHODIA)³ | 1.5% a.m. |
| DC Q2-5220 (DOW CORNING)⁴ | 1% a.m. |
| Demineralized water | Qs 100% |

¹(A) Polyurethane in an aqueous dispersion formed from 8.7% of N-methyl diethanol amine, 23.4% of isophorone diisocyanate, 67.9% of KRASOL LBH2000 (polybutadiene with hydroxyl end functions), and neutralized up to 40% using hydrogen chloride.
²Amodimethicone
³Gelling agent (hydroxypropyl guar)
⁴Water-dispersible silicone glycol

With the compositions prepared in the examples, good cosmetic properties were obtained without substantially reducing the hair fixation and hold as time goes. “Strand-effect” hair style with short hair and long-lasting hair plastering down with African hair were obtained using these compositions. They were easily removable with shampoo.

A similar result was obtained with a (B) polyurethane in an aqueous dispersion formed from 8.4% of poly(tetramethylene oxide), 8.6% of N-methyl diethanol amine, 21.4% of isophorone diisocyanate, 61.6% of KRATON L2203 (polybutadiene with hydroxyl end functions), and neutralized up to 40% using hydrogen chloride.

What is claimed is:

1. A cosmetic composition comprising, in a cosmetically acceptable aqueous medium:
(i) at least one cationic polyurethane comprising at least one non-ionic unit derived from at least one polymer chosen from olefinic homopolymers and copolymers, and
(ii) at least one silicone chosen from polydialkyl siloxanes and organomodified polysiloxanes comprising at least one functional group chosen from poly(oxyalkylene), amine and alkoxy groups.

2. A cosmetic composition according to claim 1, wherein at least 50% by weight of the polyurethane non-ionic units, relative to the total weight of the polyurethane non-ionic units are derived from at least one polymer chosen from olefinic homopolymers and copolymers.

3. A cosmetic composition according to claim 1, wherein all the polyurethane non-ionic units are derived from at least one polymer chosen from olefinic homopolymers and copolymers.

4. A cosmetic composition according to claim 1, wherein said olefinic homopolymers and copolymers are homopolymers and copolymers carrying labile hydrogen functions at their ends, and comprising units chosen from ethylene, propylene, 1-butylene, 2-butylene, isobutylene, 1,2-butadiene, 1,4-butadiene, isoprene units and mixtures thereof.

5. A cosmetic composition according to claim 4, wherein said olefinic homopolymers and copolymers are derived from optionally hydrogenated 1,2- and/or 1,4-butadiene.

6. A cosmetic composition according to claim 1, wherein the at least one cationic polyurethane comprises:
(a) cationic units resulting from the reaction of at least one tertiary or quaternary amine comprising at least two labile hydrogen-containing reactive functions,
(b) non ionic units, at least one unit (b1) of which results from the reaction of at least one polymer chosen from olefinic homopolymers and copolymers carrying labile hydrogen-containing reactive functions at their ends and having a glass transition temperature (Tg) lower than 10° C., and
(c) units resulting from the reaction of at least one diisocyanate.

7. A cosmetic composition according to claim 6, wherein the cationic (a) units result from the reaction of at least one tertiary or quaternary amine comprising two labile hydrogen-containing reactive functions.

8. A cosmetic composition according to claim 7, wherein said amine is chosen from amines having the following formulas: 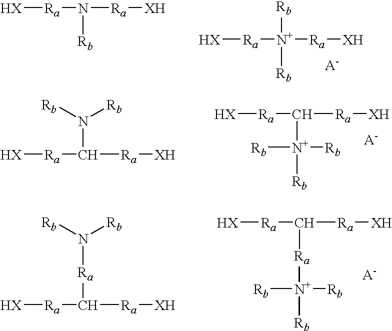
wherein
each Rₐ is independently chosen from linear or branched C₁-C₆ alkylene groups, C₃-C₆ cycloalkylene groups, arylene groups, and mixtures thereof; wherein all of them may be substituted with at least one halogen atom and comprise at least one heteroatom chosen from O, N, P and S,
each R_{b} is independently chosen from C₁-C₆ alkyl groups, C₃-C₆ cycloalkyl groups, aryl groups, and mixtures thereof; wherein all of them may be substituted with at least one halogen atom and comprise at least one heteroatom chosen from O, N, P and S,
each X is independently chosen from oxygen and sulfur atoms and from NH and NR_{c} groups, wherein R_{c} is a C₁-C₆ alkyl group, and
A⁻ is a physiologically acceptable counter-ion.

9. A cosmetic composition according to claim 8, wherein the cationic (a) units result from the reaction of N-methyldiethanol amine or N-tert-butyldiethanol amine.

10. A cosmetic composition according to claim 7, wherein the (a) units result from the reaction of at least one tertiary and/or quaternary amine function-containing polymer, carrying labile hydrogen-containing reactive functions at their ends chosen from —OH, —NH₂, —NHR_{c} and —SH, and having a weight average molecular weight ranging from 400 to 10,000, wherein R_{c} is a C₁-C₆ alkyl group.

11. A cosmetic composition according to claim 6, wherein the at least one cationic polyurethane optionally comprises at least one non-ionic (b2) unit, different from the at least one (b1) unit, derived from a non-ionic monomer compound comprising at least two labile hydrogen functions that can react with said at least one (c) compound(s) comprising at least one diisocyanate.

12. A cosmetic composition according to claim 11, wherein
the cationic (a) units are present in an amount ranging from 0.1 to 90% by weight relative to the total weight of the cationic polyurethane total units, the nonionic units derived from a (b1) olefinic homo- or copolymer are present in an amount ranging from 10 to 99.9% by weight relative to the total weight of the cationic polyurethane total units, and
the (b2) non ionic units are present in an amount ranging from 0 to 50% by weight relative to the total weight of the cationic polyurethane total units.

13. A cosmetic composition according to claim 6, wherein said at least one diisocyanate is chosen from methylenediphenyl diisocyanate, methylenecyclohexane diisocyanate, isophorone diisocyanate, toluene diisocyanate, naphthalene diisocyanate, 1,4-butane diisocyanate and 1,6-hexane diisocyanate.

14. A cosmetic composition according to claim 13, wherein said diisocyanate is isophorone diisocyanate.

15. A cosmetic composition according to claim 6, wherein the (c) units are present in an amount ranging from 1 to 60% by weight relative to the weight of the cationic polyurethane total units.

16. A cosmetic composition according to claim 6, wherein said at least one non-ionic monomer compound forming the at least one (b2) non-ionic unit(s) is chosen from C₁-C₁₂ diols and C₁-C₆ aminoalcohols

17. A cosmetic composition according to claim 6, wherein the cationic polyurethane does not comprise any further unit in addition to the (a), (b) and (c) units.

18. A cosmetic composition according to claim 6, wherein the at least one cationic polyurethane is of the elastic type.

19. A composition according to claim 1, wherein said at least one cationic polyurethane is present in an amount ranging from 0.01% to 40% by weight relative to the total weight of the composition.

20. A composition according to claim 19, wherein said at least one cationic polyurethane is present in an amount ranging from 0.1 to 10% by weight relative to the total weight of the composition.

21. A composition according to claim 1, wherein said polydialkyl siloxanes are cyclic, linear or branched polydialkyl siloxanes.

22. A composition according to claim 1, wherein said polydialkyl siloxanes are polydimethyl siloxanes that have been organomodified with poly(oxyalkylene) groups, optionally substituted amine groups, and alkoxy groups.

23. A composition according to claim 1, wherein said at least one silicone is present in an amount ranging from 0.01 to 20% by weight relative to the total weight of the composition.

24. A composition according to claim 23, wherein said at least one silicone is present in an amount ranging from 0.1 to 5% by weight relative to the total weight of the composition.

25. A composition according to claim 1, wherein said polydialkyl siloxanes are polydialkyl(C₁-C₈)siloxanes.

26. A composition according to claim 1, further comprising at least one additive chosen from gelling agents and/or thickeners, surfactants, organic solvents, fragrances, mineral, vegetable and/or synthetic oils, fatty acid esters, pH stabilizing agents, preserving agents and UV absorbers.

27. A composition according to claim 1, further comprising a gas propellant and being in the form of an aerosol.

28. A hairstyling method, comprising:
applying onto the hair a composition comprising:
(i) at least one cationic polyurethane comprising at least one non-ionic unit derived from at least one polymer chosen from olefinic homopolymers and copolymers, and
(ii) at least one silicone chosen from polydialkyl siloxanes and organomodified polysiloxanes comprising at least one functional group chosen from poly(oxyalkylene), amine and alkoxy groups;
optionally rinsing the hair, and
styling and drying the hair.
